(19) United States
(12) Patent Application Publication
(10) Pub. No.: US 2003/0109678 A1
(43) Pub. Date: Jun. 12, 2003

(54) Methods and means for regulation of gene expression

(76) Inventors: Riccardo Cortese, Massimiliano Massimo (IT); Gennaro Ciliberto, Piazzale R. Ardigo (IT); Carlo Toniatti, Benedetto Croce (IT)

Correspondence Address: MERCK AND CO INC, P O BOX 2000, RAHWAY, NJ, 070650907

(21) Appl. No.: 10/312,170

(22) PCT Filed: Jun. 15, 2001
(86) PCT No.: PCT/EP01/06792

(30) Foreign Application Priority Data
Jun. 20, 2000 (GB) 0015119.1

(51) Int. Cl. C07K 14/72; C07H 21/04; C12N 9/22; C12P 21/02; C12N 5/06

(52) U.S. Cl. 530/358; 435/69.1; 435/199; 435/320.1; 435/325; 536/23.2

(57) ABSTRACT

A transcription factor, which is a transcriptional activator or a transcriptional repressor, comprising a DNA-binding domain and a transcriptional activator or repressor domain, and optionally a regulatory domain for ligand-dependent DNA binding and/or transcriptional activation or repression by the transcription factor, wherein the transcription factor is chimeric, comprising a HNF1 polypeptide DNA-binding domain and a transcriptional activator or repressor domain of a different polypeptide, with the proviso that where the transcription factor is a transcriptional activator comprising a transcriptional activator domain the transcription factor does not comprise a regulatory domain which binds AcylHSL or an analogue thereof whereby upon AcylHSL binding DNA binding function of the DNA-binding domain is activated. A transcriptional activator comprises a human HNF1 polypeptide DNA-binding domain, a human estrogen receptor alpha regulatory domain containing a G521R mutation, and a human p65 activation domain.

The present invention relates to transcription factors useful for controlling transgenes delivered to tissues. More particularly, it relates to the use of the DNA binding domain (DBD) of HNF1 transcription factors (such as HNF1 and vHNF1) for generating chimeric transcription factors with reducing immunogenicity, useful for delivery of transgenes to tissues not expressing endogenous HNF1 or vHNF1. The present invention also relates to nucleic acid molecules and proteins useful for regulating the expression of genes in eukaryotic cells and organisms. Constitutively active as well as ligand-dependent transactivators and transrepressors containing HNF1 DBD are provided.

In the regulatory system of the invention, transcription of a nucleotide sequence is activated by a transcriptional activator fusion protein composed by the mammalian HNF1 DNA binding domain, which binds with high selectivity to selected DNA sequences, fused to different polypeptides responsible for the ligand-dependent activity of the transactivator and its transcriptional activity. The fusion proteins of the invention are useful for modulating the level of transcription of any target gene linked to the selected HNF1 DNA binding sites. The fusion proteins can be used to specifically activate transcription from genes controlled by HNF1 responsive promoters in tissues lacking endogenous HNF1 and vHNF1 proteins, such as muscles, brain, pancreas and lung. The fusion proteins of the invention are composed exclusively of mammalian elements and these may be derived from human proteins: fully human proteins mitigate the risk of immune recognition of the transactivator. Repressors are also provided in similar fashion.

An important problem encountered in the development of gene therapy in humans is the regulation of the therapeutic gene expression. To this end several regulatory systems have been developed. In general, these systems comprise three elements: (1) a target gene i.e. the gene whose expression needs to be regulated, operatively linked to a specific DNA target sequence; (2) a gene coding for a regulatory protein, i.e. a protein that regulates the activity of the target gene, generally comprising a transcriptional activation domain (AD) operatively linked to a regulatory molecule-controlled DNA binding domain (DBD), capable to bind to the DNA target sequence upon complexing with the regulatory molecule; (3) a regulatory molecule, preferably of small molecular weight, that can be added to the system from outside. For example, the relevant regulatory molecule may be added to the cells culture media or introduced in the body of the animal.

To date the four systems most commonly used to regulate gene expression are the tetracycline-dependent system (Gossen, M. and Bujard, H., 1992, Proc. Natl. Acad. Sci. USA 89:5547-5551; Gossen M. et al., 1995, Science, 268:1766-1769), the RU486-dependent system based on the use of steroid hormone receptor and the progesterone antagonist RU486 (Mifepristone or Mifegyne, Wang Y. et al., 1994, Proc. Natl. Acad. Sci. USA, 91:8180-8184), the ecdysone (Ec) dependent-system (No D. et al., 1996, Proc. Natl. Acad. Sci. USA, 93:3346-3351) and the rapamycin-dependent system (Rivera V. M. et al., 1996, Nature Med., 2:1028-1032).

These all include prokaryotic or non-human elements which are therefore likely to be immunogenic in humans or other immunocompetent hosts.

In the Tet-system, the natural Tet-controlled DNA binding domain (DBD) of the

E. coli Tet-repressor (TetR) is fused to a heterologous transcriptional activation domain (AD), usually herpes virus VP16; transcription of genes cloned downstream of a minimal promoter and TetR binding sequences can thus be controlled by tetracycline or its analogues such as doxycycline. The original Tet-off system, in which the drug de-activates transcription (Gossen, M. and Bujard, H. (1992) Proc. Natl. Acad. Sci. USA 89:5547-5551) has been superceded by the Tet-on system, in which the drug activates transcription (Gossen M. et al., 1995, Science, 268:1766-1769).

In the case of the EcB-dependent system, the natural Ec-dependent DBD from the Drosophila Ec receptor is coupled to VP16; this chimera is co-expressed with another steroid receptor (RXR) to obtain Ec-dependent transcription (No D. et al., 1996, Proc. Natl. Acad. Sci. USA, 93:3346-3351).

A more “humanized” system has been generated based on the human progesterone receptor. A carboxy-terminal deletion mutant of the human progesterone receptor was identified, which no longer binds the progesterone while still retaining the capacity of binding the synthetic progesterone antagonists RU486 (Wang Y. et al., 1994, Proc. Natl. Acad. Sci. USA, 91:8180-8184). An artificial transcription factor was constructed in which the ligand binding domain (LBD) of this mutant was fused to the DBD of the yeast transcription factor GAL4 and to VP16 activation domain. This chimera was activated by RU486 but non by progesterone and induced transcription from genes controlled by GAL-4 responsive promoters in vitro and in vivo (Wang Y. et al., 1994, Proc. Natl. Acad. Sci. USA, 91:8180-8184). In a more recent development of the system, the AD of the human p65 protein has been used as a substitute for the VP16 AD (Burcin M. M. et al., 1999, Proc. Natl. Acad. Sci. USA, 96: 355-360; Abruzzese R. V. et al., Hum. Gene Ther, 10:1499-1507).

Another partially humanized system has been generated by taking advantage of dimerising properties of rapamycin and its analogues. In this system, the transcription factor is based on a heterodimer. One monomer consists of the DNA-binding domain of the non-mammalian protein ZFHD-1 fused to the human protein FKBP12; the second is composed of another human protein, FRAP, fused to the AD of the human p65 protein (Rivera V. M. et al., 1996, Nature Med., 2:1028-1032; Rivera V. M. et al., 1999, Proc. Natl. Acad. Sci. USA, 96:8657-8662). Both FRAP and FKBP12 bind to rapamycin. Thus, in the presence of rapamycin, a heterodimeric transcription factor is formed, allowing rapamycin-dependent transcription from promoters containing ZFHD-1 recognition sites.

The present invention relates to nucleic acid molecules and proteins which can be used to regulate the expression of genes in eukaryotic cells or animals. Regulation of gene expression by the system of the invention involves at least two components: a sequence to be transcribed and optionally translated (if it encodes a protein) which is operably or operatively linked to a regulatory sequence and a protein which binds to the regulatory sequence and regulates transcription of the gene.

The present invention pertains to the use of the DBDs of liverenriched human transcription factors HNF1 for generating fully transcription factors, preferably “humanized” transcription factors likely to have reduced immunogenicity in humans. A transcription factor according to the invention activates or represses transcription when bound to an HNF1-dependent promoter comprising HNF1 binding sites.

Thus, according to one aspect of the present invention there is provided a transcription factor, which may be a transcriptional activator or repressor, composed of a DNA-binding domain and a transcriptional activator or repressor domain, and optionally a regulatory domain for ligand-dependent DNA binding and/or transcriptional activation or repression by the transcription factor, wherein the DNA-binding domain is of a HNF1 polypeptide and the transcriptional activator or repressor domain is of a different polypeptide. This may be with the proviso that where the transcription factor is a transcriptional activator comprising a transcriptional activator domain the transcription factor does not comprise a regulatory domain which binds AcylHSL or an analogue thereof whereby upon AcylHSL binding DNA binding function of the DNA-binding domain is activated.

Preferably a human HNF1 DNA binding domain is used in conjunction with a human transcriptional activator or repressor domain, and optionally a human regulatory domain, i.e. the relevant domain of a human polypeptide.

The repressor or activator domain and the DNA-binding domain may be provided within a fusion protein, which may additionally comprise a regulatory domain. Alternatively, the DNA-binding domain and a regulatory domain may be provided within a fusion protein, which may associate with a transcriptional activator or repressor domain to provide a functional transcription factor.

Suitable regulatory domains are discussed further below. LuxR regulatory domains and others that are responsive to N-acyl-homoserine-lactone (AcylHSL) may not be employed in certain embodiments of the invention (especially where the transcription factor is a transcriptional activator). Thus, LuxR-type transcription factor may be excluded, i.e. homologues of the Vibrio fischeri LuxR protein (Fuqua, et al.; 1994; J. Bacteriol., 176:269-275). According to the general teaching of Henikoff, S., et al (Henikoff, S. Wallace, J C. Brown, J P., 1990; Methods Enzimol. 183: 111-132) and more specifically of Fuqua, et al (Fuqua, et al.; 1994; J. Bacteriol., 176:269-275; Fuqua, C., et al; 1996; Annu. Rev. Microbiol., 50:727-751), members of a LuxR superfamily of LuxR-type transcription factor are defined by the following characteristics:

(1) are DNA-binding proteins that are a component of an N-acyl homoserine lactone based gene regulatory system;

(2) comprise a first cluster of sequence similarity in a region that aligns with the putative AcylHSL-binding region of LuxR.

(3) their carboxyl terminal thirds comprise a second cluster of sequence similarity in a region defined as a helix-turn-helix motif contained within the DNA binding domain. This helix-turn-helix motif is identified as containing a motif defined as a probe helix putatively involved in protein-DNA major groove interaction in a number of transcription factors (Suzuki, M. 1993; EMBO J., 8: 3221-3226). Sequence similarity is generally recognised using the ExPASY public server of the Swiss Institute of Bioinformatics. A signature pattern defining LuxR family membership, defined by PROSITE (Protein Family and Domain Database of the Swiss Institute of Bioinformatics, 1, Rue Michel-Servet, 1211, Genève, 4 Switzerland) is the following:

[GDC]-x(2)-[NSTAVY]-x(2)-[IV]-[GSTA]-x(2)-[LIVMFYWCT]-x[LIVMFYWCR]-x(3)-[NST]-[LIVM]-x(5)-[NRHSA]-[LIVMSTA]-x(2)-[KR].

Addition LuXR signature patterns may be defined with reference to the “Blocks” database at the Fred Hutchinson Cancer Research Center in Seattle, Wash., USA or at the Weizmann Institute of Science in Israel.

The HNF1 DNA-binding domain is able to bind an HNF1 binding site within a nucleic acid molecule, specifically within a promoter region to provide for transcriptional activation or repression by means of the relevant transcriptional activation or repression domain.

Unless differently specified, throughout this application the acronym HNF1 is intended to include any known form of HNF1, such as HNF1, vHNF1-A and vHNF1-B, and by “HNF1 binding site” is intended any specific binding site for any of the known forms of HNF1 (see below for references).

Natural HNF1 polypeptides are transcription factors expressed at high levels in hepatocytes and responsible for the transcription of several liver-specific genes, such as albumin and alpha1-antitrypsin. They are also expressed in tissues other than liver, such as kidney, intestine, stomach and pancreas. However, HNF1 proteins are not naturally expressed in several cell lines and tissues. In particular, the fact that HNF1 are not expressed in muscles is of relevance for gene therapy purposes in accordance with the present invention.

Direct intramuscular injection of either viral- or non-viral vectors is one of the preferred modes for transgene delivery in vivo. In particular, direct intramuscular injection of viral- or non-viral vectors encoding: i) antigens from viruses, bacteria or protozoans result in the protection against a subsequent challenge with the corresponding pathogen; ii) tumor-specific antigens result in protection of mice against challenges with tumorigenic cells expressing the corresponding antigen; iii) secreted proteins result in delivery into the bloodstream (Marshall, D. J. and Leiden, J. M., 1998, Curr. Opin. Genet. Dev., 8, 360-365).

A transgene cloned downstream of an HNF1-dependent promoter is not transcribed when delivered in cells lacking endogenous HNF1 (Toniatti C. et al., 1990, EMBO J., 9, 4467-4475). Since HNF1 are not present in muscles, a transgene cloned downstream of an HNF1-dependent promoter may be silent when delivered into muscle cells in vivo and in vitro. However, previous results obtained in vitro provide indication that such a transgene could be activated if an expression vector encoding for HNF1 is co-delivered into muscles (Toniatti C. et al., 1990, EMBO J., 9, 4467-4475).

Different functional domains of HNF1 are known in the art (Chouard T. et al., 1990, Nucleic Acids Res, 18, 5853-5863; Nicosia A. et al., 1990, Cell, 1225-1236, Toniatti C. et al., 1993, DNA and Cell Biology, 12, 199-208).

HNF1 (also called LF-B1 or HNF1alpha) is a 628 aa long protein DNA binding protein that has been implicated as a major determinant of hepatocyte-specific transcription of several genes (Frain M. 1990, Cell, 59, 145-157). The consensus binding site derived from these sequences is the palindrome GGTTAAT(N)ATTAATA (Tronche F. et la., 1997, J. Mol Biol., 266:231-245). Consistent with the dyad symmetry of this site, HNF1 binds DNA as a dimer. The functional domains of HNF1 have been dissected by site directed mutagenesis (Chouard T. et al., 1990, Nucleic Acids Res, 18, 5853-5863; Nicosia A. et al., 1990, Cell, 1225-1236, Toniatti C. et al., 1993, DNA and Cell Biology, 12, 199-208): the residues required for transcriptional activity of the molecule are located in the C-terminal part (aa 282-628), whereas the DNA binding activity maps in the first N-terminal 281 aa (DBD=1-281). Within the DNA binding domain of HNF1, three regions have been identified, namely A, B and C (Nicosia A. et al., 1990, Cell, 1225-1236). Region A (aa 1-32) has been shown to be necessary and sufficient to bring about dimerization of the protein through an α-helical structure (De Francesco R. et al., 1991, Biochemistry, 30, 143-147; Pastore A. et al., 1991, Biochemistry, 30, 148-153). Region B (aa 100-184) and region C (aa. 198-281) show limited homology respectively to the POU-A box and to the POU-homeodomains of POU proteins (Herr W., 1988, Genes Dev., 2, 1513-1516; REF). The homeodomain-like structure of the HNF1 DBD has an insertion of 21 aa between helix II and helix III, as compared to canonical homeobox (Finney, M., 1990, Cell, 60-5-6; Ceska T. A., 1993, EMBO J., 12, 1805-1810).

A protein with a strong primary sequence homology to HNF1 has also been cloned (Rey-Campos J., 1991, EMBO J., 10, 1445-1457; De Simone V. et al., 1991, EMBO J., 10, 1435-1443) and called variant-HNF1 (vHNF1) or LF-B3 or HNF1beta. HNF1 and vHNF1 share strong homology at the amino acid level in their DBD (A, B, and C regions; Rey-Campos J., 1991, EMBO J., 10, 1445-1457; Frain M. 1990, Cell, 59, 145-157). The sequence homology between HNF1 and vHNF1 declines toward the C-terminal part of the sequences, where the AD has been mapped. In rat, mouse and human two different cDNAs coding for vHNF1 are generated by an alternative splicing and have been called vHNF1A and vHNF1B. vHNF1A is 559 amino acids long and contains an extra 26 aa long segment that is absent in vHNF1B, which is 533 aa long. This sequence is located between the B-domain and the C-domain of the DBD and is also absent in HNF1. In the present application, we refer to vHNF1A and vHNF1B collectively with the name vHNF1.

In line with the fact that vHNF1 and HNF1 DBDs share strong sequence homology, the two proteins have the same DNA binding specificity and are capable of forming heterodimers in solution and on DNA (Tronche F. et la., 1997, J. Mol Biol., 266:231-245). During mouse or rat development, vHNF1 expression systematically precedes HNF1 expression (Lazzaro D. et al., 1992, Development, 114, 469-479; Cereghini, S., 1992, Development, 116, 783-797). Although these proteins are believed to be responsible for the transcription of several liver specific genes, they both are expressed also in tissues other than liver, such as kidney, intestine, stomach and pancreas. HNF1 mRNA was also detected in spleen and testis while vHNF1 mRNA was also detected in lung and ovary (De Simone V. et al., 1991, EMBO J., 10, 1435-1443; Blumenfeld M., 1991, Development, 113, 589-599; Emens L. A. et al., 1992, Proc. Natl. Acad. Sci. USA, 89, 7300-7304).

The present inventors have for the first time employed HNF1 DNA binding domain to construct functional chimeric transcription factors comprising either activation or repressor domains other than HNF1, optionally with ligand-dependent regulatory domains. The transactivators of the present invention can therefore be used to specifically and effectively regulate transcription from co-delivered transgene cloned downstream of HNF1 responsive promoters in cells and tissues that do not express endogenous HNF1.

The terms “chimeric” and “chimera” are used herein with reference to fusion proteins and transcription factors, activators and repressors of the invention, to denote composition of components of different origin, in particular of different parent proteins. Thus a transcription factor composed of HNF1 DBD and p65 AD (see below) is considered chimeric. This is irrespective of any inter-species chimericity, and indeed in preferred embodiments a chimeric transcription factor of the invention is composed only of human protein components.

For the development of vectors useful in veterinary gene therapy, and to control gene expression in non-human mammalian cells, HNF1 DNA binding domain of animals rather than human origin can be used. The invention is not limited to human HNF1 DBD but further pertains to any chimeric transcription factor that comprises the HNF1/vHNF1 DBDs of mammalian species other than human.

A transcriptional activator including a fusion protein according to the present invention may comprise a portion of a naturally occurring HNF1/vHNF1 protein, of which examples have been mentioned. Furthermore, one or more of the polypeptide components may be employed which comprise an amino acid sequence which differs by one or more amino acid residues from the known natural amino acid sequence, whether a mutant, allele, isoform, variant or derivative of a specific sequence. Instead of using a wild-type DBD, a transcriptional activator according to the present invention may include a DBD whose amino acid sequence differs by one or more amino acid residues from the wild-type amino acid sequence, by one or more of addition, insertion, deletion and substitution of one or more amino acids but still retains the same binding specificity.

Preferably, the amino acid sequence shares homology with a fragment of the relevant protein, preferably at least about 30%, or 40%, or 50%, or 60%, or 70%, or 75%, or 80%, or 85%, 90% or 95% homology. Thus, a protein component may include 1, 2, 3, 4, 5, greater than 5, or greater than 10 amino acid alterations such as substitutions with respect to the wild-type sequence.

As is well-understood, homology at the amino acid level is generally in terms of amino acid similarity or identity. Similarity allows for “conservative variation”, i.e. substitution of one hydrophobic residue such as isoleucine, valine, leucine or methionine for another, or the substitution of one polar residue for another, such as arginine for lysine, glutamic for aspartic acid, or glutamine for asparagine. Similarity may be as defined and determined by the TBLASTN or other BLAST program, of Altschul et al., (1990) J. Mol. Biol. 215, 403-10, which is in standard use in the art, or, and this may be preferred, either of the standard programs BestFit and GAP, which are part of the Wisconsin Package, Version 8, September 1994, (Genetics Computer Group, 575 Science Drive, Madison, Wis., USA, Wisconsin 53711). BestFit makes an optimal alignment of the best segment of similarity between two sequences. Optimal alignments are found by inserting gaps to maximize the number of matches using the local homology algorithm of Smith and Waterman (Advances in Applied Mathematics (1981) 2, pp. 482-489). GAP uses the Needleman and Wunsch algorithm to align two complete sequences that maximizes the number of matches and minimizes the number of gaps. Generally, the default parameters are used, with a gap creation penalty=12 and gap extension penalty=4. Homology is generally over the full-length of the relevant sequence compared with the relevant wild-type amino acid sequence.

A further way of defining similarity or identity between sequences is to consider ability of nucleic acid to hybridize under stringent conditions. As noted further below, a fusion protein and polypeptide components thereof according to the present invention are generally provided by expression from encoding nucleic acid. Such encoding nucleic acid may be employed in hybridization experiments.

Preliminary experiments may be performed by hybridizing under low stringency conditions. Preferred conditions are those which are stringent enough for there to be a simple pattern with a small number of hybridizations identified as positive which can be investigated further.

For example, hybridizations may be performed, according to the method of Sambrook et al. (below) using a hybridization solution comprising: 5×SSC (wherein “SSC”=0.15 M sodium chloride; 0.15 M sodium citrate; pH 7), 5× Denhardt's reagent, 0.5-1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 1×SSC and 1% SDS; (4) 2 hours at 42-65° C. in 1×SSC and 1% SDS, changing the solution every 30 minutes.

One common formula for calculating the stringency conditions required to achieve hybridization between nucleic acid molecules of a specified sequence homology is (Sambrook et al., 1989): Tₘ=81.5° C.+16.6Log[Na+]+0.41 (% G+C)−0.63 (% formamide)−600/#bp in duplex.

As an illustration of the above formula, using [Na+]=[0.368] and 50-% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

It is well known in the art to increase stringency of hybridization gradually until only a few positive clones remain. Other suitable conditions include, e.g. for detection of sequences that are about 80-90% identical, hybridization overnight at 42° C. in 0.25M Na₂HPO₄, pH 7.2, 6.5% SDS, 10% dextran sulfate and a final wash at 55° C. in 0.1×SSC, 0.1% SDS. For detection of sequences that are greater than about 90% identical, suitable conditions include hybridization overnight at 65° C. in 0.25M Na₂HPO₄, pH 7.2, 6.5% SDS, 10% dextran sulfate and a final wash at 60° C. in 0.1×SSC, 0.1% SDS.

As noted, the invention provides a transcription factor which comprises: (1) a first polypeptide component that binds in a sequence specific manner to an operator sequence in DNA—this being a DNA-binding domain of an HNF1 polypeptide; and (2) a second polypeptide component that activates or represses transcription in eukaryotic cells. The transcription factor may additionally comprise a regulatory domain which binds a cognate ligand whereby upon binding of regulatory domain and ligand DNA binding function of the DNA binding domain is altered, and/or transcriptional activation or repression is activated or repressed.

Regulation of gene expression by the system of the invention involves at least two components: a nucleic acid sequence which is operably or operatively linked to an HNF1-dependent promoter, and a chimeric transcription factor comprising at least one DNA binding domain of HNF1 and which binds to the promoter sequence to modulate transcription of the gene.

In a regulatory system in accordance with the invention, transcription of a nucleotide sequence is activated by a transcriptional activator composed of at least two polypeptide components: (i) an HNF1 DNA-binding domain; (ii) a transcriptional activating or repressing domain; and optionally at least (iii) a ligand-dependent regulatory domain.

In a preferred embodiment of the invention the HNF1 DNA binding domain comprises or consists of residues 1-282 of human HNF1 (Bach, et al (1990), Genomics, 8(1):155-164 (Sequence accession number P20823), or a DNA-binding portion encompassed within these residues; in another preferred embodiment the HNF1 DNA binding domain comprises or consists of residues 1-314 of human vHNF1A, or a DNA-binding portion encompassed within these residues; in another preferred embodiment the HNF1 DNA binding domain comprises or consists of residues 1-289 of vHNF1B (ReyCampos J., 1991, EMBO J., 10, 1445-1457; De Simone V. et al., 1991, EMBO J., 10, 1435-1443), or a DNA-binding portion encompassed within these residues.

In an aspect of the invention the transcriptional activator may comprise the HNF1 DNA binding domain fused to a transcriptional activator domain, which activates transcription in eukaryotic cells, either directly or indirectly. Transcriptional activators according to this aspect of the invention are constitutively active.

Otherwise, the transcription factor of the invention may be conditionally active, and may comprise a ligand-dependent regulatory domain as discussed further below.

The transcriptional activator or repressor domains may be any available to those skilled in the art. Polypeptides which activate transcription in eukaryotic cells are well known in the art. In particular, transcriptional activation domains of many DNA binding proteins have been described and have been shown to retain their activation function when the domain is transferred to a heterologous protein.

In a preferred embodiment of the invention the transcriptional activator domain is an activation domain (AD) of human p65 protein (Schmitz, M. L. and Bauerle, P. A., 1991, EMBO J., 10:3805-3817), more preferably comprising or consisting of the region spanning amino acids 283-551 of human p65, or a transcription-activating portion encompassed within this region. In another embodiment, multimers of the p65 AD may be used. In another embodiment, multimers of portions of the p65 AD may be used.

In another preferred embodiment of the invention the transcriptional activator comprises the herpes simplex virus virion protein 16 (referred to herein as VP16, the amino acid sequence of which is disclosed in Triezenberg, S. J. et al. (1988) Genes Dev. 2:718-729), more preferably about 127 of the C-terminal amino acids of VP16 are used; more preferably about 11 of the C-terminal amino acids (amino acids 437-447) of VP16 are used. Preferably, multimers (two to four monomers) of this region are used. Preferably, a dimer of this region (i.e., about 22 amino acids) is used. Suitable C-terminal peptide portions of VP16 are described in Seipel, K. et al. (EMBO J., 1992 13:4961-4968). For example, a dimer of a peptide having an amino acid sequence DALDDFDLDML can be used.

In another embodiment of the invention the transcriptional activator comprises or consists of the AD of the PPARγ-1 coactivator (PGC-1) whose sequence is disclosed in Puigserver P. et al., 1998, Cell, 92, 829. In one embodiment, the region spanning aa 1-170 of the N-terminus of PGC-1 is used (Puigserver, P., Science, 1999, 1368-1371). In another embodiment, the region spanning aa 1-65 of the N-terminus of PGC-1 is used. In another embodiment, multimers of the PGC-1 AD may be used. In another embodiment, multimers of portions of the PGC-1 AD may be used.

In other embodiments, chimeric transcription factors capable of repressing transcription are generated (Transcriptional Repressors). In this case, the transcription factor comprises a repressor domain, which directly or indirectly repress transcription in eukaryotic cells. An example of such a domain, capable of repressing instead of activating transcription, is the KRAB repressor domain of the human Kox1 zinc finger protein (Margolin J., 1994, Proc. Natl. Acad. Sci. USA, 91: 4509-4513).

Other polypeptides with transcriptional activation ability in eukaryotic cells can be used in a transcriptional activator in accordance with the invention. Transcriptional activation domains found within various proteins have been grouped into categories based upon similar structural features. Types of transcriptional activation domains include acidic transcription activation domains, proline-rich transcription activation domains, serine/threonine-rich transcription activation domains and glutamine-rich transcription activation domains. Examples of acidic transcriptional activation domains include the VP16 regions already described and amino acid residues 753-881 of GAL4. Examples of proline-rich activation domains include amino acid residues 399-499 of CTF/NF1 and amino acid residues 31-76 of AP2. Examples of serine/threonine-rich transcription activation domains include amino acid residues 1-427 of ITF1 and amino acid residues 2-451 of ITF2. Examples of glutamine-rich activation domains include amino acid residues 175-269 of Oct1 and amino acid residues 132-243 of Sp1. The amino acid sequences of each of the above described regions, and of other useful transcriptional activation domains, are disclosed in Seipel, K. et al. (EMBO J., 1992 12:4961-4968).

The transcriptional activation ability of a polypeptide can be assayed by linking the polypeptide to another polypeptide having DNA binding activity and determining the amount of transcription of a target sequence that is stimulated by the fusion protein. For example, a standard assay used in the art utilizes a fusion protein of a putative transcriptional activation domain and a GAL4 DNA binding domain (e.g., amino acid residues 1-93). This fusion protein is then used to stimulate expression of a reporter gene linked to GAL4 binding sites (see e.g., Seipel, K et al., 1992 EMBO J. 11:4961-4968 and references cited therein).

Transcriptional repressors domain, which directly or indirectly repress transcription in eukaryotic cells, can be used in the invention. An example of such domains, capable of repressing instead of activating transcription, is the KRAB repressor domain of the human Koxl zinc finger protein (Margolin J., 1994, Proc. Natl. Acad. Sci. USA, 91,4509-4513). This domain can be used either as single domain or in multimeric forms.

Polypeptides which repress transcription in eukaryotic cells are well known in the art. In particular, transcriptional repression domains of many DNA binding proteins have been described and have been shown to retain their activation function when the domain is transferred to a heterologous protein (Deuschle et al., 1995, Mol. Cell. Biol. 15, 1907-1914; Freundlieb S. et al., 1999, J. Gene Medicine, 1, 1).

A ligand-dependent regulatory domain is a domain of a transactivator or transrepressor that regulates the activity of the transactivator or transrepressor molecule upon binding to a specific ligand. Best known example of such regulatory domains are the ligand binding domains (LBD) of the steroid receptors. Steroid receptors are transcription factors that activate transcription only upon binding, via their LBD, to their cognate ligand.

An example of ligand-dependent regulatory domain is the mutated form of the human estrogen receptor alpha containining a G521R mutation: it displays a reduced affinity for estradiol while maintaining a high affinity for synthetic estrogen antagonists, such as 4-hydroxytamoxifen (4-OHT), a metabolite of Tamoxifen (TAM), and several others (e.g., raloxifen) (Danelian, P. S. et al., 1993, Mol. Endocrinol., 7:232-240; Feil R. et al, 1996, Proc. Natl. Acad. Sci. USA, 93:10887-10890).

Another example is the RU486-depedent C-terminal deletion of the human progesterone receptor, which does not bind progesterone but only its synhtetic analog RU486 (Vegeto E. et al., 1992, Cell, 69:703-713).

Regulatory domains can also be constructed by using protein domains that heterodimerize in the presence of a specific ligand. For instance, the HNF1 DBD can be fused to the rapamycin binding domain of human protein FKBP12. This chimera will dimerize, in the presence of rapamycine, with a second drug-binding domain, such as that of FRAP protein, fused to a human transcriptional regulatory domain. This rapamycine-mediated dimer will be able to activate transcription from an HNF1 responsive promoter.

In all cases described, the concentration of active transcription factor (i.e comprising both the DNA binding domain and the transcriptional regulatory domain) will be proportional to the concentration of the ligand.

The term ligands encompasses compounds which need not be structurally related to steroid but which can be used as ligand for the regulatory domains of the chimeric transcription factors.

In another aspect of the invention, a chimeric transcription factor comprises HNF1 DBD and (i) a polypeptide comprising a ligand-dependent regulatory domain (for instance, the LBD of a steroid receptor or a mutant form, responsible for binding steroids, steroid agonists and antagonists), (ii) a transcriptional activator domain, which directly or indirectly activates transcription in eukaryotic cells. The activity of transcription factors according to this aspect of the invention, and as consequence the transgene transcription, can be selectively modulated by adding or withdrawing the specific ligand.

In a preferred embodiment of the invention a ligand-dependent transcription factor comprising the DBD of HNF1, is constructed as a tamoxifen-dependent chimera. This chimera is constituted by the DNA binding domain of HNF1 fused to a mutated form (Gly 521 to Arg) of the human estrogen receptor α (Danelian, P. S. et al., 1993, Mol. Endocrinol., 7:232-240; Feil R. et al, 1996, Proc. Natl. Acad. Sci. USA, 93:10887-10890) and the p65 activation domain. The mutated form of the human estrogen receptor binds estradiol with a strongly reduced affinity but retains high affinity for estradiol antagonists, such as 4-hydroxytamoxifen (4-OHT), a metabolite of Tamoxifen (TAM), and others (e.g. raloxifen) (Danelian, P. S. et al., 1993, Mol. Endocrinol., 7:232-240; Feil R. et al, 1996, Proc. Natl. Acad. Sci. USA, 93:10887-10890).

In another embodiment of the invention an RU486-dependent transactivator is employed, by fusing HNF1 DBD in frame with a C-terminal deletion of the human progesterone receptor, which does not bind progesterone but only its synthetic analog RU486 (Vegeto E. et al., 1992, Cell, 69:703-713), followed by an in-frame transcriptional activator or repressor domain.

In other embodiments, chimeric proteins capable of repressing transcription are generated (Transcriptional Repressors). In one embodiment, the fusion protein comprises (i) HNF1 DBD linked to (ii) a mutated form (Gly 521 to Arg) of the human estrogen receptor a (Danelian, P. S. et al., 1993, Mol. Endocrinol., 7:232-240; Feil R. et al, 1996, Proc. Natl. Acad. Sci. USA, 93:10887-10890) linked to (iii) the KRAB repressor domain of the human Koxl zinc finger protein (Margolin J., 1994, Proc. Natl. Acad. Sci. USA, 91,4509-4513).

It should be noted that the three essential components of the ligand binding-dependent transcripton factors, namely the DNA binding domain, the ligand-dependent regulatory domain and the transcriptional regulatory domain, may be arranged in any order or sequence in a transactivator/transrepressor fusion protein of the invention.

In another embodiment, the HNF1 DBD and the transcriptional regulatory domain can be provided separately, as two separate fusion proteins, whereby said fusion proteins interact in order to provide an active transcription factor. For instance, the mammalian HNF1 DBD may. be fused to a ligand-binding domain (for instance, FKBP12) which can dimerise, in the presence of the ligand (for instance rapamycin), with another ligand-binding domain (for instance FRAP) fused to a human transcriptional regulatory domain. In this case the concentration of active transcription factor (i.e comprising both the DNA binding domain and the transcriptional regulatory domain) will be proportional to the concentration of the ligand.

In another embodiment, transcription is activated by an indirect mechanism, through recruitment of a transcriptional activation protein to interact with a fusion protein comprising DBD and regulatory domain. This may, for example, be via a polypeptide domain (e.g., a dimerization domain) which mediates a proteinprotein interaction with a transcriptional activator protein, such as an endogenous activator present in a host cell. Examples of suitable interaction (or dimerization) domains include leucine zippers (Landschulz et al. (1989) Science 243:1681-1688), helix-loop-helix domains (Murre, C. et al. (1989) Cell 58:537-544) and zinc finger domains (Frankel, A. D. et al. (1988) Science 240:70-73).

A transcription factor of the invention (which may be a single fusion protein) may further comprise one or more additional polypeptide components, such as a fourth polypeptide component which promotes transport into a cell nucleus, a nuclear localization signal (NLS). Nuclear localization signals typically are composed of a stretch of basic amino acids. When attached to a heterologous protein (e.g., a fusion protein of the invention), the nuclear localization signal promotes transport of the protein to a cell nucleus. The nuclear localization signal is attached to a heterologous protein such that it is exposed on the protein surface and does not interfere with the function of the protein. Preferably, the NLS is attached to one end of the protein, e.g. the N-terminus. The amino acid sequence of a non-limiting example of an NLS that can be included in a fusion protein of the invention is Met-Pro-LysArg-Pro-Arg-Pro. Preferably, a nucleic acid encoding the nuclear localization signal is spliced by standard recombinant DNA techniques in-frame to the nucleic acid encoding the fusion protein (e.g., at the 5′ end).

Transcription factors containing the DBD of the invention specifically activate (or repress) transcription of sequences controlled by HNF1 responsive promoters. Fusion proteins containing the HNF1 DBD are useful for regulating, in tissues that do not express endogenous HNF1, the level of transcription of any target gene linked to the selected HNF1 DNA binding sites.

HNF1 dependent promoters may comprise single or mutimeric HNF1 binding sites.

For use in embodiments of the invention, HNF1 dependent promoters may comprise at least one HNF1 binding site and one or more binding sites for one or more different transcription factors.

In preferred embodiments, an HNF1-based activator is used to activate transcription from an artificial HNF1 dependent promoter comprising one or multiple HNF1 binding sites (e.g. two, three, four, five, six, seven, eight, nine, ten or more HNF1/vHNF1 binding sites).

In other preferred embodiments, an HNF1-based repressor is used to repress transcription from a constitutively active promoter which also comprises one or more natural or artificially introduced HNF1 binding sites. These promoters are constitutively active in the absence of HNF1 transcription factors, but are specifically repressed by HNF1 based repressors.

The invention is widely applicable to a variety of situations where it is desirable to be able to turn gene expression on and off, or regulate the level of gene expression. The only prerequisite is that the target cells or tissues do not contain active endogenous HNF1-based transcription factors.

The invention is preferentially employed for gene therapy purposes, e.g. in treatments for genetic or acquired diseases, especially in those cases in which a long-term treatment is required and avoiding an immune response against the transactivator is preferable. (e.g. therapy of genetic and chronic diseases).

To use a system for gene therapy purposes in accordance with the present invention, cells of a subject in need of gene therapy may be modified to contain (1) nucleic acid encoding an HNF1based transactivator or transrepressor in a form suitable for expression in the host cells and (2) a sequence of interest (e.g. for therapeutic purposes) operatively linked to an HNF1/vHNF1 dependent promoter.

Where an HNF1-based ligand-dependent activator is employed, expression of the sequence of interest in cells of the subject is stimulated by administering the relevant ligand/inducing agent to the patient. To stop expression of the gene of interest in cells of the subject, administration of the inducing agent is stopped.

Where an HNF1-based ligand-dependent repressor is employed, 10 expression of the sequence of interest in cells of the subject is repressed in the presence of the ligand and then stimulated by its withdrawal. To stop expression of the gene of interest in cells of the subject, the ligand is readministered.

In both cases the level of gene expression can be modulated by adjusting the dose of the ligand administered to the patient. Thus, in a host cell, transcription of a sequence operatively linked to an HNF1-dependent promoter may be controlled by altering the concentration of the inducer ligand (for instance, TAM, 4-OHT or other steroids and their analogues) in contact with the host cell (e.g. adding the ligand to a culture medium, or administering the ligand to a host organism, etc.).

To induce or repress transcription in vivo the ligand may be administered to the body, or a tissue of interest (e.g. by injection). The body to be treated may be that of an animal, particularly a mammal, which may be human or non-human, such as rabbit, guinea pig, rat, mouse or other rodent, cat, dog, pig, sheep, goat, cattle or horse, or which is a bird, such as a chicken. Suitable routes of administration include oral, intraperitoneal, intramuscular, i.v.

As noted, the invention provides for construction of regulatory systems that have the advantage over other available regulatory systems of minimising the risk of immunogenicity, in that HNF1 DBD of human origin can be used, thus allowing to construct fully humanised transactivators.

Besides the use for gene therapy outlined in the previous sections, ligand-dependent transcription factors incorporating the HNF1 DBD of the invention can be used to:

1 conditionally express a suicide gene in cells, thereby allowing for elimination of the cells after they have served an intended function. For example, cells used for vaccination can be eliminated in a subject after an immune response has been generated by the subject by inducing expression of a suicide gene in the cells with the specific ligand.

2 modulate expression of genes that are contained in recombinant viral vectors and might interfere with the growth of the viruses in the packaging cell lines during the production processes. These recombinant viruses might be derivatives of Adenoviruses, Retroviruses, Lentiviruses, Herpesviruses, Adenoassociated viruses and other viruses which are familiar and obvious to those skilled in the art.

3 provide large scale production of a toxic protein of interest using cultured cells in vitro that do not contain endogenous HNF1/vHNF1 and which have been modified to contain a nucleic acid encoding the transactivator carrying the DBD of the invention in a form suitable for expression of the transactivator in the cells and a gene encoding the protein of interest operatively linked to an HNF1-dependent promoter.

One convenient way of producing a polypeptide or fusion protein according to the present invention is to express nucleic acid encoding it, by use of nucleic acid in an expression system.

Accordingly the present invention also provides in various aspects nucleic acid encoding the transcriptional activator or repressor of the invention, which may be used for production of the encoded protein.

Generally whether encoding for a protein or component in accordance with the present invention, nucleic acid is provided as an isolate, in isolated and/or purified form, or free or substantially free of material with which it is naturally associated, such as free or substantially free of nucleic acid flanking the gene in the human genome, except possibly one or more regulatory sequence(s) for expression. Nucleic acid may be wholly or partially synthetic and may include genomic DNA, cDNA or RNA. Where nucleic acid according to the invention includes RNA, reference to the sequence shown should be construed as encompassing reference to the RNA equivalent, with U substituted for T.

Nucleic acid sequences encoding a polypeptide or fusion protein in accordance with the present invention can be readily prepared by the skilled person using the information and references contained herein and techniques known in the art (for example, see Sambrook, Fritsch and Maniatis, A Molecular Cloning, A Laboratory Manual, Cold Spring Harbor Laboratory Press (1989), and Ausubel et al., Current Protocols in Molecular Biology, John Wiley and Sons, (1994)), given the nucleic acid sequence and clones available. These techniques include (i) the use of the polymerase chain reaction (PCR) to amplify samples of such nucleic acid, e.g. from genomic sources, (ii) chemical synthesis, or (iii) preparing cDNA sequences. DNA encoding portions of full-length coding sequences (e.g. a DNA binding domain, or regulatory domain as the case may be) may be generated and used in any suitable way known to those of skill in the art, including by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Modifications to the relevant sequence may be made, e.g. using site directed mutagenesis, to lead to the expression of modified peptide or to take account of codon preference in the host cells used to express the nucleic acid.

In order to obtain expression of the nucleic acid sequences, the sequences may be incorporated in a vector having one or more control sequences operably linked to the nucleic acid to control its expression. The vectors may include other sequences such as promoters or enhancers to drive the expression of the inserted nucleic acid, nucleic acid sequences so that the polypeptide or peptide is produced as a fusion and/or nucleic acid encoding secretion signals so that the polypeptide produced in the host cell is secreted from the cell. Polypeptide can then be obtained by transforming the vectors into host cells in which the vector is functional, culturing the host cells so that the polypeptide is produced and recovering the polypeptide from the host cells or the surrounding medium. Prokaryotic and eukaryotic cells are used for this purpose in the art, including strains of E. coli, yeast, and eukaryotic cells such as COS or CHO cells.

Thus, the present invention also encompasses a method of making a polypeptide or fusion protein as disclosed, the method including expression from nucleic acid encoding the product (generally nucleic acid according to the invention). This may conveniently be achieved by growing a host cell in culture, containing such a vector, under appropriate conditions which cause or allow expression of the polypeptide. Polypeptides may also be expressed in in vitro systems, such as reticulocyte lysate.

Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others. A common, preferred bacterial host is

E. coli.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. phage, or phagemid, as appropriate. For further details see, for example, Molecular cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

For use in mammalian cells, a recombinant expression vector's control functions may be provided by viral genetic material. Exemplary promoters include those derived from polyoma, Adenovirus 2, cytomegalovirus and SV40.

A regulatory sequences of a recombinant expression vector used in the present invention may direct expression of a polypeptide or fusion protein preferentially in a particular cell type, i.e., tissue-specific regulatory elements can be used. In one embodiment, the recombinant expression vector of the invention is a plasmid. Alternatively, a recombinant expression vector of the invention can be a virus, or portion thereof, which allows for expression of a nucleic acid introduced into the viral nucleic acid. For example, replication defective retroviruses, adenoviruses and adeno-associated viruses can be used. Protocols for producing recombinant retroviruses and for infecting cells in vitro or in vivo with such viruses can be found in Ausubel, et al. (supra). The genome of a virus such as adenovirus can be manipulated such that it encodes and expresses a transactivator or repressor protein but is inactivated in terms of its ability to replicate in a normal lytic viral life cycle.

Thus, a further aspect of the present invention provides a host cell containing heterologous nucleic acid as disclosed herein.

The host cell can be, for example, a mammalian cell (e.g., a human cell), a yeast cell, a fungal cell or an insect cell. Moreover, the host cell can be a fertilized non-human oocyte, in which case the host cell can be used to create a transgenic organism having cells that express the transcriptional inhibitor fusion protein. Still further, the recombinant expression vector can be designed to allow homologous recombination between the nucleic acid encoding the transactivator or repressor and a target gene in a host cell. Such homologous recombination vectors can be used to create homologous recombinant animals that express a fusion protein of the invention.

The nucleic acid of the invention may be integrated into the genome (e.g. chromosome) of the host cell. Integration may be promoted by inclusion of sequences which promote recombination with the genome, in accordance with standard techniques. The nucleic acid may be on an extra-chromosomal vector within the cell, or otherwise identifiably heterologous or foreign to the cell.

Examples of mammalian cell lines which may be used include CHO dhfr-cells (Urlaub and Chasin (1980) Proc. Natl. Acad Sci. USA 77:4216-4220), 293 cells (Graham et al. (1977) J. Gen. Virol. 36: pp 59) and myeloma cells like SP2 or NS0 (Galfre and Milstein (1981) Meth. Enzymol. 73(B):3-46). In addition to cell lines, the invention is applicable to normal cells, such as cells to be modified for gene therapy purposes or embryonic cells modified to create a transgenic or homologous recombinant animal. Examples of cell types of particular interest for gene therapy purposes include hematopoietic stem cells, myoblasts, hepatocytes, lymphocytes, muscle cells, neuronal cells and skin epithelium and airway epithelium. Additionally, for transgenic or homologous recombinant animals, embryonic stem cells and fertilized oocytes can be modified to contain nucleic acid encoding a transactivator or repressor fusion protein.

Nucleic acid a transactivator or repressor fusion protein can transferred into a fertilized oocyte of a non-human animal to create a transgenic animal which expresses the fusion protein of the invention in one or more cell types.

Aspects of the invention further provide non-human transgenic organisms, including animals, that contains cells which express transcriptional activator or repressor protein of the invention (i.e., a nucleic acid encoding the transactivator or repressor is incorporated into one or more chromosomes in cells of the transgenic organism).

A still further aspect provides a method which includes introducing the nucleic acid into a host cell. The introduction, which may (particularly for in vitro introduction) be generally referred to without limitation as “transformation”, may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. As an alternative, direct injection of the nucleic acid could be employed.

Marker genes such as antibiotic resistance or sensitivity genes may be used in identifying clones containing nucleic acid of interest, as is well known in the art.

The introduction may be followed by causing or allowing expression from the nucleic acid, e.g. by culturing host cells (which may include cells actually transformed although more likely the cells will be descendants of the transformed cells) under conditions for expression of the gene, so that the encoded product is produced. If the polypeptide is expressed coupled to an appropriate signal leader peptide it may be secreted from the cell into the culture medium. Following production by expression, a polypeptide may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers (e.g. see below).

Introduction of nucleic acid encoding a polypeptide according to the present invention may take place in vivo by way of gene therapy. One option is to introduce nucleic acid into cells ex vivo, which cells may then be implanted or otherwise administered to an individual. Such cells may have been taken from the individual and may be returned after treatment with nucleic acid of the invention.

Thus, a host cell containing nucleic acid according to the present invention, e.g. as a result of introduction of the nucleic acid into the cell or into an ancestor of the cell and/or genetic alteration of the sequence endogenous to the cell or ancestor (which introduction or alteration may take place in vivo or ex vivo), may be comprised (e.g. in the soma) within an organism which is an animal, particularly a mammal, which may be human or non-human, such as rabbit, guinea pig, rat, mouse or other rodent, cat, dog, pig, sheep, goat, cattle or horse, or which is a bird, such as a chicken. Genetically modified or transgenic animals and birds comprising such a cell are also provided as further aspects of the present invention.

A host cell containing a transcriptional activator or repressor of the invention (e.g. a fusion protein provided by transformation of the host cell with encoding nucleic acid) may additionally contain (e.g. as a result of transformation) one or more nucleic acids which serve as a target for the transcriptional activator. A target nucleic acid comprises a nucleotide sequence to be transcribed operatively linked to at least one operator sequence.

A transcriptional activator or repressor in accordance with the present invention may be used to regulate transcription of a target nucleotide sequence which is operatively or operably linked to a regulatory sequence to which the transcriptional activator or repressor binds. The nucleotide sequence to be transcribed typically includes a minimal promoter sequence which is not itself transcribed but which serves (at least in part) to position the transcriptional machinery for transcription. The minimal promoter sequence is located upstream of the transcribed sequence to form a contiguous nucleotide sequence. The activity of such a minimal promoter is dependent upon the binding of a transcriptional activator or repressor to an operatively linked regulatory operator sequence. The minimal promoter may be from the human cytomegalovirus (as described in Boshart et al. (1985) Cell 41:521-530), and other suitable minimal promoters are available to those skilled in the art.

The target nucleotide sequence is operatively linked to at least one oligonucleotide sequence to which a transcriptional activator of the invention binds, an HNF1 operator sequence. The operator is usually 5′ to the sequence to be transcribed and, where appropriate, minimal promoter. An operator sequence may be operatively linked downstream (i.e., 3′) of the nucleotide sequence to be transcribed.

The further sequence operably linked to the promoter and operator sequences may be a coding sequence for a polypeptide or peptide, an antisense sequence or a ribozyme.

A polypeptide of which expression may be controlled using the present invention may be selected according to the desires and aims of the person performing the invention, and may be a therapeutic protein or a cytotoxic protein.

Polypeptide expression may be inhibited by using appropriate nucleic acid to influence expression by antisense regulation, and an antisense sequence may be placed under transcriptional control in accordance with the present invention. The use of anti-sense genes or partial gene sequences to down-regulate gene expression is now well-established. Double-stranded DNA is placed under the control of a promoter in a “reverse orientation” such that transcription of the “anti-sense” strand of the DNA yields RNA which is complementary to normal mRNA transcribed from the “sense” strand of the target gene. The complementary anti-sense RNA sequence is thought then to bind with mRNA to form a duplex, inhibiting translation of the endogenous MRNA from the target gene into protein. Whether or not this is the actual mode of action is still uncertain. However, it is established fact that the technique works.

Another possibility is that nucleic acid is used which on transcription produces a ribozyme, able to cut nucleic acid at a specific site—thus also useful in influencing gene expression. Background references for ribozymes include Kashani-Sabet and Scanlon (1995). Cancer Gene Therapy, 2, (3) 213-223, and Mercola and Cohen (1995). Cancer Gene Therapy 2, (1) 47-59.

A transcription unit of the invention may be incorporated into a recombinant vector (e.g., a plasmid or viral vector), and may be introduced into a host cell or animal, optionally along with a transcriptional activator as disclosed or encoding nucleic acid therefor.

A further aspect of the present invention provides a composition comprising:

(i) a transcriptional activator as disclosed, or a first nucleic acid encoding a transcriptional activator as disclosed; and

(ii) a second nucleic acid comprising a nucleotide sequence to be transcribed operatively linked to a transcription unit.

In one embodiment, where both a first and a second nucleic acid are included, the first and second nucleic acids are separate molecules (e.g., two different vectors). In this case, a host cell may be cotransfected with the two nucleic acid molecules or successively transfected first with one nucleic acid molecule and then the other nucleic acid molecule. Furthermore, the components of a trancriptional activator comprising a fusion protein which comprises DBD and ligand-binding components and another polypeptide providing transcriptional activation or repression which interacts with the fusion to provide a transactivator or transrepressor may be provided as separate molecules. In another embodiment, the nucleic acids are linked (i.e., colinear) in the same molecule (e.g., a single vector). In this case, a host cell may be transfected with the single nucleic acid molecule.

The invention further provides a method of treatment which includes administering to a patient an agent which comprises (i) a transcription factor according to the invention, or nucleic acid encoding such a fusion protein, and/or (ii) a transcription unit as disclosed. The invention further provides for use of such components (i) and (ii) in the manufacture of a medicament for administration to an individual.

A transcriptional activator or repressor according to the present invention may be used to regulate transcription of a sequence by means of an operator sequence operably linked to the sequence to be transcribed. As discussed, this operator/transcribed sequence construct may be introduced into host cells. In an alternative, a sequence to be transcribed may be endogenous to a host cell. An endogenous sequence may be operatively linked to an appropriate transcription unit by means of homologous recombination. For example, a homologous recombination vector can be prepared which includes at least one HNF1 operator sequence and a miminal promoter sequence flanked at its 3′ end by sequences representing the coding region of the endogenous gene and flanked at its 5′ end by sequences from the upstream region of the endogenous gene by excluding the actual promoter region of the endogenous gene. The flanking sequences are of sufficient length for successful homologous recombination of the vector DNA with the endogenous gene. Preferably, several kilobases of flanking DNA are included in the homologous recombination vector. Upon homologous recombination between the vector DNA and the endogenous gene in a host cell, a region of the endogenous promoter is replaced by the vector DNA containing one or more HNF1 operator sequences operably linked to a minimal promoter. Thus, expression of the endogenous gene is no longer under the control of its endogenous promoter but rather is placed under the control of the transcription unit in accordance with the present invention.

In another embodiment, an operator sequence may be inserted elsewhere within an endogenous gene, preferably within a 5′ or 3′ regulatory region, via homologous recombination to create an endogenous gene whose expression can be regulated by a transcriptional activator or repressor described herein. For example, one or more HNF1 binding sequences can be inserted into a promoter or enhancer region of an endogenous gene such that promoter or enhancer function is maintained.

The term “HNF1 binding site” or “HNF1 binding sequence” is meant a natural or artificial DNA sequence that is bound by HNF1/vHNF1 transactivators (Tronche F., 1997, J. Mol. Vol., 266, 231-245). A nucleotide sequence to be transcribed can be operatively linked to an HNF1/vHNF1 dependent promoter which can be constituted by one single or multiple HNF1/vHNF1 binding sites (e.g., two, three, four, five, six, seven, eight, nine, ten or more HNF1/vHNF1 binding sites) mixed or not with binding sites for other transcription factors.

Chimeric promoters can be used, wherein at least one HNF1 binding site is linked to at least one binding site for another transcriptional factor.

A composition according to the present invention that is to be given to an individual, administration is preferably in a “prophylactically effective amount” or a “therapeutically effective amount” as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors. A composition may be administered alone or in combination with other treatments, either simultaneously or sequentially dependent upon the condition to be treated.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Liposomes, particularly cationic liposomes, may be used in carrier formulations.

Examples of techniques and protocols mentioned above can be found in Remington's Pharmaceutical Sciences, 16th edition, Osol, A. (ed), 1980.

The agent may be administered in a localised manner to a tumour site or other desired site or may be delivered in a manner in which it targets tumour or other cells.

Targeting therapies may be used to deliver the active agent more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

Instead of administering these agents directly, they may be produced in the target cells by expression from an encoding gene introduced into the cells, e.g. in a viral vector. The vector may targeted to the specific cells to be treated, or it may contain regulatory elements which are switched on more or less selectively by the target cells.

A composition may be administered alone or in combination with other treatments, either simultaneously or sequentially dependent upon the condition to be treated, such as cancer, virus infection or any other condition in which an effect mediated by activity of the fusion protein is desirable.

Nucleic acid according to the present invention, encoding a transcriptional activator or repressor may be used in methods of gene therapy, for instance in treatment of individuals, e.g. with the aim of preventing or curing (wholly or partially) a disorder or for another purpose as discussed elsewhere herein.

Vectors such as viral vectors have been used in the prior art to introduce nucleic acid into a wide variety of different target cells. Typically the vectors are exposed to the target cells so that transfection can take place in a sufficient proportion of the cells to provide a useful therapeutic or prophylactic effect from the expression of the desired polypeptide. The transfected nucleic acid may be permanently incorporated into the genome of each of the targeted cells, providing long lasting effect, or alternatively the treatment may have to be repeated periodically.

A variety of vectors, both viral vectors and plasmid vectors, are known in the art, see U.S. Pat. No. 5,252,479 and WO 93/07282. In particular, a number of viruses have been used as gene transfer vectors, including papovaviruses, such as SV40, vaccinia virus, herpesviruses, including HSV and EBV, and retroviruses. Many gene therapy protocols in the prior art have used disabled murine retroviruses.

As an alternative to the use of viral vectors other known methods of introducing nucleic acid into cells includes electroporation, calcium phosphate co-precipitation, mechanical techniques such as microinjection, transfer mediated by liposomes and direct DNA uptake and receptor-mediated DNA transfer.

Receptor-mediated gene transfer, in which the nucleic acid is linked to a protein ligand via polylysine, with the ligand being specific for a receptor present on the surface of the target cells, is an example of a technique for specifically targeting nucleic acid to particular cells.

### BRIEF DESCRIPTION OF THE DRAWINGS

FIG. 1 shows a schematic structure of estrogen receptor α.

FIG. 2 shows a schematic structure of 4-OHT-dependent transcription factor HEA-1.

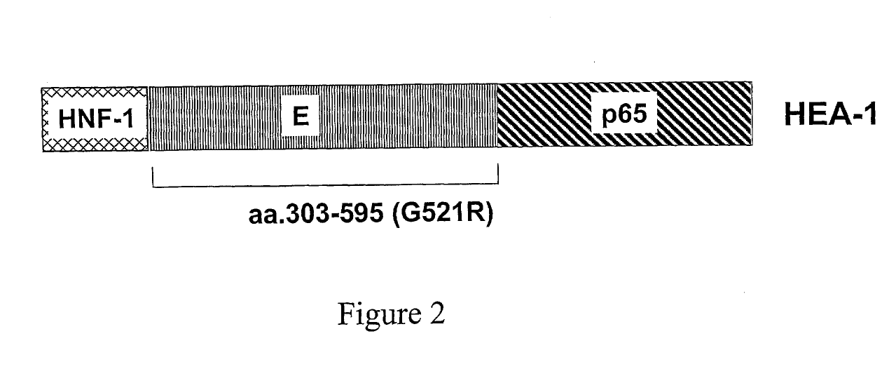

FIG. 3 shows a schematic structure of reporter mEpoI gene used in vitro and in vivo for testing the transcriptional properties of HEA-1.

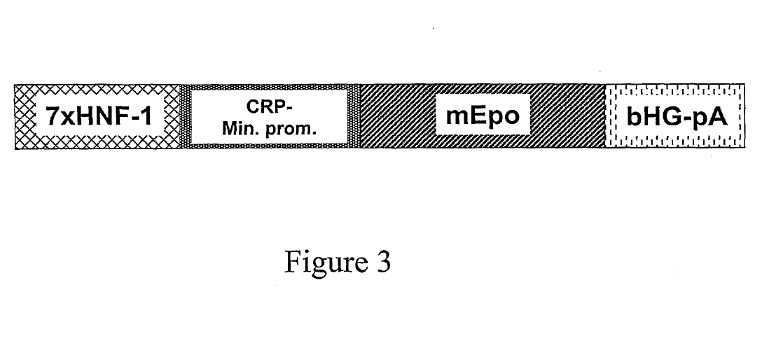

FIG. 4 illustrates in vitro 4-OHT, TAM and E2 responsiveness of HEA-1, as measured by production of mEpo. mEpo activity (mU/ml) is plotted against nM hormone for E2 (lower squares) TAM (triangles) and 4-OHT (upper squares).

FIG. 5 illustrates in vivo results obtained by using HEA-1 and mEpo-1 in mice. Open squares are for mEpo1/HEA (5 μg/1 μg): w/o TAM; closed squares for mEpo1/HEA-1 (5 μg/1 μg): w/TAM.

FIG. 6 shows the schematic structure of constructs HEA-3 and HEA-4.

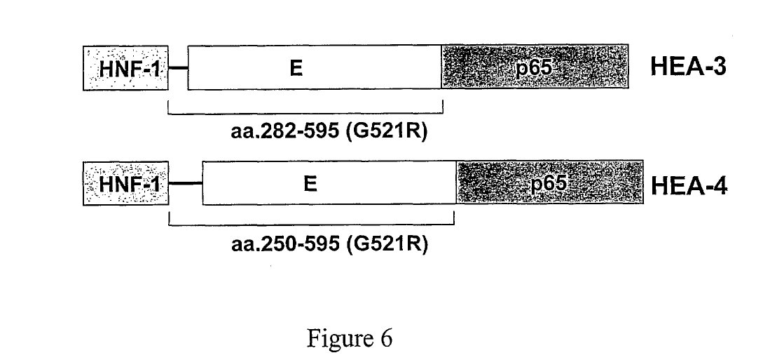

FIG. 7 shows the results of experiments demonstrating in vivo 4-OHT and E2 responsiveness of HEA-1, HEA-3 and HEA-4. Circles: open—HEA-1 (E2), closed—HEA-1 (4-OHT); squares: open—HEA-3 (E2), closed HEA-3 (4-OHT); triangles: open—HEA-4 (E2), closed—HEA4 (4-OHT). HEA-3 displays the highest activity and inducibility.

FIG. 8 shows results of experiments demonstration longevity of regulation using HEA-3 in vivo, with hematocrit (%) plotted against time in days. Closed squares are for mEpo-1/HEA-3 (1 μg/1 μg): w/TAM (1 mg/kg). Open squares are for mEpo-1HEA-3 (1 μg/1 μg): w/o TAM.

FIG. 9 shows results of in vivo experiments demonstrating reversibility of induction. Hematocrit (%) is plotted against time in days, for mEpo-1/HEA-3 (1 μg/1 μg): w/ and w/o TAM (1 mg/kg).

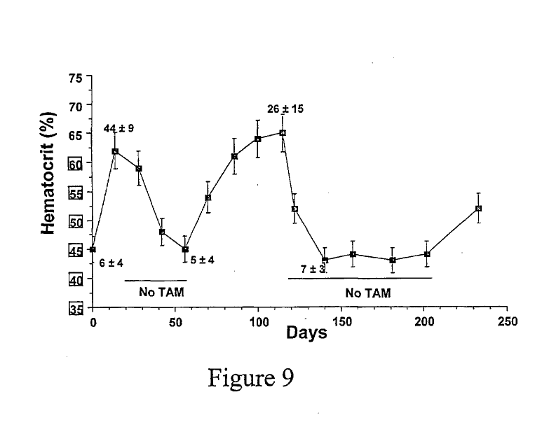

### EXPERIMENTAL

The examples below are provided as a further guide to the skilled person, and are not to be constructed as limiting the invention in any way. Further aspects and embodiments will be apparent to those skilled in the art.

### EXAMPLE 1

Construction of HEA-1

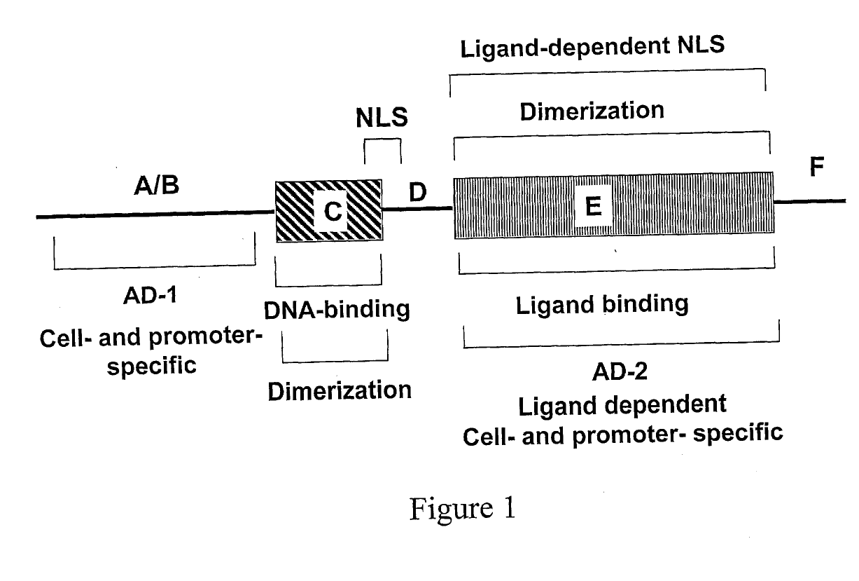

As an example of the possibility of generating ligand-dependent transcription factors comprising the DBD of HNF1/vHNF1, the inventors constructed a tamoxifen-dependent chimera. This chimera, called HEA-1, is constituted by the DNA binding domain of HNF1 fused to a mutated form of the human estrogen receptor a (FIG. 1), and the p65 AD. The mutated form of the human estrogen receptor (ER), contains a G521R mutation: it displays an at least 1,000 fold-reduced affinity for Estradiol (E2) as compared to wt ER but efficiently binds synthetic derivatives, such as 4-hydroxytamoxifen (4-OHT), a metabolite of Tamoxifen (TAM), and several others (e.g., raloxifen). The inventors tested the capability of this transactivator to activate in a ligand-dependent manner the transcription of genes cloned downstream of HNF1-depedent promoters in vitro and in vivo. In vivo experiments were done by electro-injecting plasmids DNA into mice muscles. The leakiness (e.g. ligand-independent transcriptional activity) of the transactivator was assessed as well as its degree of inducibility. HEA-1 was obtained by in-frame fusion of nucleic acids encoding the human HNF1 DNA binding domain, the mutated LBD of the human ERa and the AD of human p65 protein according to standard clonig technique (Ausubel, F. M. et al., 1995, Current Protocols in Molecular Biology, John Wiley & Sons, New York, N.Y.). This chimeric protein is constituted from its N-terminus to its C-terminus by the following elements: the DBD of human HNF-1 (aa. 1-282, [Frain M. 1990, Cell, 59, 145-157; Nicosia A. et al., 1990, Cell, 1225-1236]), a linker constituted by two aa. (D-I, Aspartate-Isoleucine), the HBD of the human estrogen receptor spanning aa. 303-595 of the human estrogen receptor and containing the G521R mutation (Danelian, P. S. et al., 1993, Mol. Endocrinol., 7:232-240; Feil R. et al, 1996, Proc. Natl. Acad. Sci. USA, 93:10887-10890) and the p65 activation domain spanning aa. 283-551 of the human NF-κB p65 protein (Burcin et al., 1999, Proc. Natl. Acad. Sci. U.S.A. 96, 355-360; Abruzzese et al., 1999, Hum. Gene Ther., 10:1499-1507). The cDNA coding for the whole protein was cloned downstream of the CMV enhancer/promoter element and the intron A sequence into plasmid pviJnsA (Montogomery D. et al., 1993, DNA Cell. Biol., 12, 777-783), thus obtaining the expression vector pCMV/HEA-1.

Plasmid pCMV/HEA-1 was constructed as follows. Nucleic acid encoding the DBD of human HNF-1 (aa. 1-282) was obtained as a PCR fragment using appropriate primers on plasmid HA (Yaniv M. 1993, EMBO J., vol 12, no. 11) which was the template of the PCR reaction: the obtained fragment was digested at its 5′ and 3′ with BglII and EcoRV restriction enzymes, respectively. The digested fragment was cloned downstream of the CMV enhancer/promoter element and the intron A sequence into plasmid pViJnsA (Montgomery D. et al., 1993, DNA Cell. Biol., 12, 777-783) digested with BglII (5′) and EcoRV (3′) restriction enzymes. The G521R ER-HBD (region 303-595 of the human estrogen receptor containing the G521R mutation) was cloned in frame with the HNF-1 DBD at the level of the EcORV site. In particular, the G521R ER-HBD was obtained by site-directed mutagenesis of the wild-type human ER-HBD in the context of the human ER-HBD contained in plasmid phERalpha(LBD)/TGEM (Zhou G. et al., 1998, Mol. Endocrinol. 12:1594-1604). The plasmid containing the G521R ER-HBD was used as the template for PCR amplification with appropriate primers and a fragment containing the G521R ER-HBD was obtained, digested at its 5′ end with EcoRV and at its 3′ end with EcoRI restriction enzymes and cloned in frame with the HNF-1 DBD at the level of the EcoRV site (located at the 3′ end of the HNF-1 DBD and the 5′ end of the G521R ER-HBD. Finally, the p65 activation domain containing the translation stop codon was obtained as an EcoRI fragment from plasmid pGS1158 (Abruzzese et al., 1999, Hum. Gene Ther., 10:1499-1507) and introduced in frame with the G512R HBD at the EcoRI site located at the 3′ of the HBD.

An HNF1 responsive promoter was constituted by multimerized HNF1 binding sites cloned upstream of a minimal promoter element. In this particular example, it was constituted by seven tandem repeats of the HNF1 binding site derived from the rat albumin promoter and the minimal promoter element of the C-reactive protein: the construction of this HNF1 responsive promoter is described in the literature (Toniatti C. et al., 1990, EMBO J., 9, 4467-4475).

The mEpo coding region was assembled from synthetic oligonucleotide as described (Rizzuto et al., 1999, Proc. Natl. Acad. Sci. USA, 96:6417-6422; Maione et al., 2000, Hum. Gene Ther. 11:859:868). The mEpo cDNA was cloned into PstI-BamHI sites of the polylinker of plasmid pBluescript II KS (Maione et al., 2000, Hum. Gene Ther. 11:859:868). The bovine growth hormone (bGH) polyadenylation sequences, derived from nucleotides 983 to 1249 of the pcDNA2 vector (InVitrogen, NV Leek, The Netherlands) was cloned into XbaI-NotI site 3′ of the mEpo cDNA, providing the polyadenylation signal. This plasmid was called pBSKS/mEpo-polyA. The HNF-1 responsive promoter was excised as an 5′-EcoRI (filled with Klenow) and 3′-HindIII fragment from plasmid 7xHNF-1/CRP-CAT (Toniatti C. et al., 1990, EMBO J., 9, 4467-4475) and cloned upstream of the mEpo gene in plasmid pBSKS/mEpo-polyA, previously digested 5′ with SalI (filled with Klenow) and 3′ with HindIII restriction enzymes. This plasmid was called pBS/7xB1/mEpo-polyA. The cassette containing the HNF-1 responsive promoter, the mEpo cDNA and the bGH polyadenylation sequences was excised from plasmid pBS/7xB1/mEpo-polyA as a 5′-KpnI(blunted with T4 exonuxlease) and 3′-NotI fragment and inserted into EcoRV-NotI sites of plasmid pViJ/PL, thus obtaining plasmid pViJ/7xB1/mEpo-polyA, which is also called plasmid mEpo-1. Plasmid pViJ/PL is a derivative of plasmid pViJnsA (Montogomery D. et al., 1993, DNA Cell. Biol., 12, 777-783) in which a polylinker sequence replaces the CMV enhancer-promoter element and the intron A originally present in plasmid pViJnsA.

### EXAMPLE 2

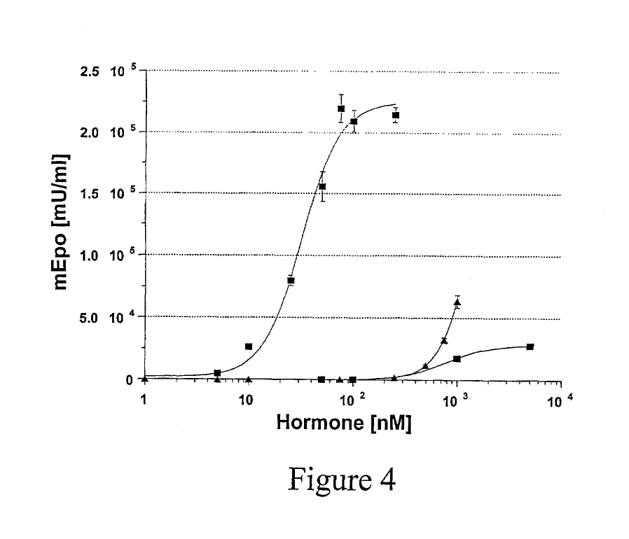

In vitro testing of HEA-1 activity In vitro testing of HEA-1 activity (FIG. 4) HEA-1 has been tested by transfection in HeLa cells which do not contain endogenous HNF-1 (Toniatti C. et al., 1990, EMBO J., 9, 4467-4475) treated or not with Estradiol (E2), Tamoxifen (TAM) or 4-hydroxytamoxifen (4-OHT). HeLa cells were propagated in Dulbecco's modified Eagle's medium supplemented with 10% fetal calf serum plus glutamine and antibiotics at 37 □C in 5% CO₂. For tranfection experiments, 3×10⁵ cells were seeded in a 60-mm-diameter dish: 20 h later they were cotransfected with 0.5 μg of CMV/HEA-1 expression vector and 4.5 μg of mEpo-1 reporter gene. Transfections were performed by using the calcium-phosphate technique (Ausubel, F. M. et al., 1995, Current Protocols in Molecular Biology, John Wiley & Sons, New York, N.Y.). At 15 h later, the cells were washed, and either E2 or TAM or 4-OHT were added at various concentrations to the culture medium. After 36 h, the culture medium was collected and the mEpo protein secreted in the culture medium was measured using a commercially available ELISA assay (R & D Systems) for human Epo cross-reactive with mEpo and know amounts of recombinant mEpo (Boehringer Manneheim) as a standard reference (Rizzuto et al., 1999, Proc. Natl. Acad. Sci. USA, 96:6417-6422). The results (FIG. 4) indicated that HEA-1 is exquisitely sensitive to 4-OHT and is only weakly activated by E2. Therefore, the LBD retains, in the context of the fusion, the expected binding properties.

### EXAMPLE 3

In vivo Testing of HEA-1 Activity

Mice muscles were electro-injected with 1 μg of an HEA-1 expression vector CMV/HEA-1 and 5 μg of the reporter plasmid containing the mEpo cDNA cloned downstream of seven tandem repeats of the HNF1 binding site and (plasmid mEpo-1). Seven-weeks old Balb/c female mice were electro-injected into quadriceps with the quantity of DNA indicated before using the electro-injection technique exactly as described in Rizzuto et al., 1999, Proc. Natl. Acad. Sci. USA, 96:6417-6422. Electroinjected mice were treated or not (4 mice for each group) with 5.6 mg/Kg of TAM, p.o., daily: Hct as well as plasma mEpo levels were monitored after 14 days. Activation of transcription of mEpo gene activated by TAM, is reflected by increased Hct in mice.

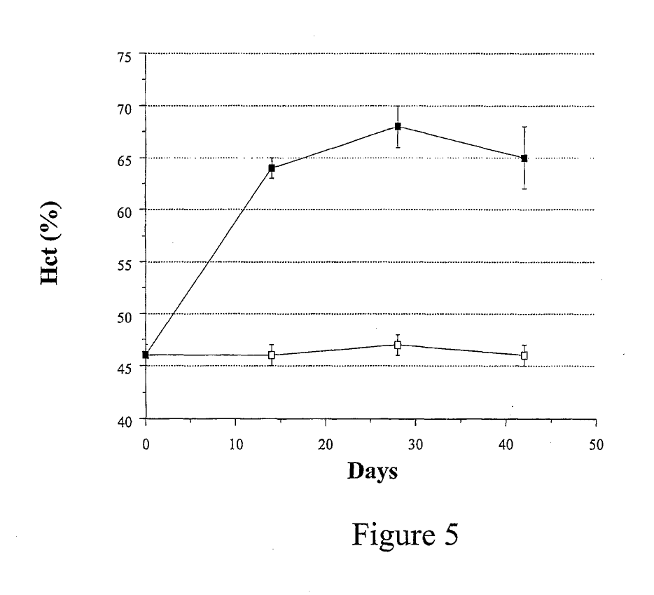

Results are shown in FIG. 5. A strong Hct increase was observed only in mice treated with 5.6 mg/Kg daily of TAM: no leakiness (i.e. TAM-independent Hct increase) was observed at the DNA quantity injected.

This indicated that (1) the HNF-1 responsive promoter is exclusively stimulated by the ligand-activated HEA-1 transcription factor, and (2) the HNF-1 responsive promoter is not activated by endogenous transcription factors other than HNF-1.

### EXAMPLE 4

Construction and in vivo testing of HEA-3 and HEA-4

HEA-1 contains the G521R mutation in the context of an ER-HBD spanning amino acids 303-595 of the receptor. Further constructs were made in which amino acid residues contained in the D region of the ER-alpha (FIG. 1) were added.

HEA-3 was constructed, which contains the G521R mutation in the context of an ER-HBD spanning amino acids 282-595, and HEA-4 which has the same mutation but in an HBD spanning amino acids 252-595. The cDNAs coding for these mutants were constructed according to the same procedure as described for HEA-1 in Example 1.

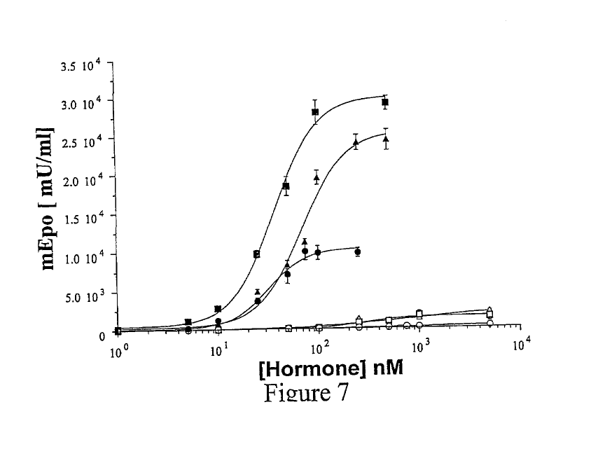

cDNAs for HEA-3 and HEA-4 were separately cloned downstream of the CMV enhancer/promoter element and intron A sequence into plasmid pV1JnsA, thus obtaining the expression vectors pCMV/HEA3 and pCMV/HEA-4. Again, the strategy was the same as used for constructing pCMV/HEA-1 (Example 1). HEA-3 and HEA-4 were then tested in vitro in HeLa cells as described in Example 2. Transfected cells were treated or not with Estradiol (E2) and 4-hydroxytamoxifen (4-OHT). The results, shown in FIG. 7, demonstrate that HEA-3 is slightly more sensitive to 4-OHT and has a higher maximal activity as compared with HEA-1 and HEA-4.

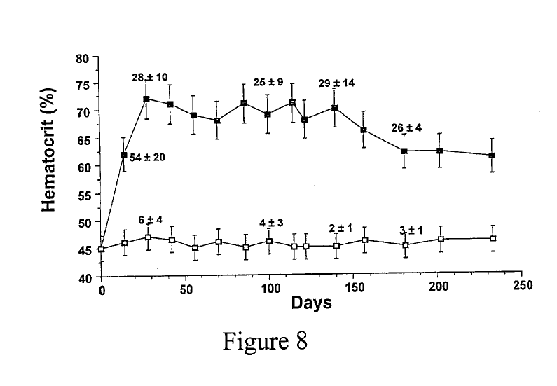

HEA-3 was then tested in vivo exactly as described for HEA-1 in Example 3. Mice muscles were electroinjected with 1 μg of pCMV/HEA-3 and 1 μg of the Epo reporter plasmid. Seven week old Balb/c female mice were electroinjected in the quadriceps with the quantities of DNA indicated (for FIG. 8) before using the electroinjection technique (see Example 3). Electroinjected mice were treated or not (4 mice for each group) with 1 mg/kg of TAM, p.o., 5 days/wee: Hct as well as plasma Epo levels were monitored every 2 weeks. Results are shown in FIG. 8 in which the Hct levels of the mice as well as the Epo levels measure at day 14, 28, 100, 140 and 180 post-injection are indicated. Notably, treated mice displayed a strong Hct increase and a significant elevation of mEpo levels up to day 240 post-injection, strongly indicating that the transactivator is not immunogenic in mice. No Hct increase was detected in untreated mice.

Reversibility of TMA-dependent induction was then tested upon TAM-withdrawal. 8 female Balb/c mice (7 weeks old) were electroinjected with 1 μg of pCMV/HEA-3 and 1 μg of the Epo reported plasmid described above. Mice were then continuously treated with TAM (1 mg/kg, p.o., 5 days/week) for 14 days. Serum Epo and Hct increased as expected. In the absence of treatment with TAM, Hct returned to basal level with a kinetic compatible with the known half-life of erythrocytes in mice (about 18 days). The hematocrit returned to high levels when these animals were challenged again with TAM, thus demonstrating that the responsiveness to the ligand is maintained over time.

All documents cited in this specification are incorporated herein by reference.

1. A transcription factor, which is a transcriptional activator or a transcriptional repressor, comprising a DNA-binding domain and a transcriptional activator or repressor domain, and optionally a regulatory domain for ligand-dependent DNA binding and/or transcriptional activation or repression by the transcription factor,
wherein the transcription factor is chimeric, comprising a HNF1 polypeptide DNA-binding domain and a transcriptional activator or repressor domain of a different polypeptide,
with the proviso that where the transcription factor is a transcriptional activator comprising a transcriptional activator domain the transcription factor does not comprise a regulatory domain which binds AcylHSL or an analogue thereof whereby upon AcylHSL binding DNA binding function of the DNA-binding domain is activated.

2. A transcription factor according to claim 1 comprising a human HNF1 DNA-binding domain and a human transcriptional activator or repressor domain, and optionally a human regulatory domain.

3. A transcription factor according to claim 1 or claim 2, wherein the transcriptional activator or repressor domain and the DNA-binding domain are comprised within a fusion protein, wherein said fusion protein optionally comprises said regulatory domain.

4. A transcription factor according to claim 1 or claim 2 wherein the DNA-binding domain and a regulatory domain are comprised within a fusion protein, which fusion protein associates with a transcriptional activator or repressor domain to form the transcription factor.

5. A transcription factor according to any one of claims 1 to 4 wherein the HNF1 polypeptide DNA-binding domain comprises residues 1-282 of human HNF1 or a DNA-binding portion encompassed within these residues.

6. A transcription factor according to any one of claims 1 to 4 wherein the HNF1 polypeptide DNA-binding domain comprises residues 1-314 of human vHNF1A, or a DNA-binding portion encompassed within these residues.

7. A transcription factor according to any one of claims 1 to 4 wherein the HNF1 polypeptide DNA-binding domain comprises residues 1-289 of vHNF1B, or a DNA-binding portion encompassed within these residues.

8. A transcription factor according to any one of claims 1 to 7 which comprises a ligand-dependent regulatory domain.

9. A transcription factor according to claim 8 wherein the ligand-dependent regulatory domain is a human estrogen receptor alpha regulatory domain containing a G521R mutation.

10. A transcription factor according to claim 9 comprising amino acids 303-595 of the human estrogen receptor containing the G521 mutation.

11. A transcription factor according to claim 10 comprising amino acids 282-595 of the human estrogen receptor containing the G521 mutation.

12. A transcription factor according to claim 11 comprising amino acids 252-595 of the human estrogen receptor containing the G521 mutation.

13. A transcription factor according to any one of claims 1 to 12 which is a transcriptional activator comprising a transcriptional activator domain.

14. A transcription factor according to claim 13 comprising the HNF1 polypeptide DNA-binding domain fused to the transcriptional activator domain.

15. A transcription factor according to claim 13 or claim 14 wherein the transcriptional activator domain is a human p65 protein activator domain.

16. A transcription factor according to claim 15 wherein the transcriptional activator domain comprises residues 283-551 of human p65, or a transcription-activating portion encompassed within these residues.

17. A transcription factor according to any one of claims 1 to 16 comprising one or more additional polypeptide components.

18. A transcription factor according to claim 17 comprising a nuclear localization signal (NLS).

19. Nucleic acid encoding a transcription factor according to any one of claims 1 to 18.

20. Nucleic acid according to claim 19 wherein the transcription factor is a fusion protein comprising the DNA-binding domain, a transcriptional activator or repressor domain, and a regulatory domain for ligand-dependent DNA binding and/or transcriptional activation or repression by the transcription factor.

21. A nucleic acid vector comprising nucleic acid according to claim 19 or claim 20.

22. A nucleic acid vector according to claim 21 wherein the nucleic acid encoding the transcription factor is under control of regulatory sequences for expression of the transcription factor.

23. A host cell transformed with a nucleic acid vector according to claim 22.

24. A method of making a transcription factor, the method comprising culturing a host cell according to claim 23 under conditions for production of the transcription factor.

25. A method of stimulating or repressing transcription, the method comprising binding a transcription factor according to any one of claims 1 to 18 to an operator sequence operatively linked to a target nucleotide sequence.

26. A method according to claim 25 wherein the transcription factor comprises a regulatory domain for ligand-dependent DNA binding and/or transcriptional activation or repression by the transcription factor, and said binding occurs within a host cell, the method comprising treating the host cell with ligand of the regulatory domain to activate binding of the transcription factor to the operator sequence.

27. A method according to claim 26 wherein said host cell is cultured in vitro in a medium containing the ligand.

28. A method according to any one of claims 25 to 27 wherein the target nucleotide sequence encodes a product polypeptide.

29. A method according to claim 28 wherein the polypeptide is produced by expression from the target nucleotide sequence, the method further comprising isolating and/or purifying the product polypeptide.

30. A method according to claim 29 wherein the product polypeptide is formulated into a composition comprising at least one additional component.

31. A method according to any one of claims 25 to 27 wherein the target nucleotide sequence provides, on transcription, an antisense sequence.

32. A method according to any one of claims 25 to 27 wherein the target nucleotide sequence provides, on transcription, a ribozyme.

33. A composition comprising:
(i) a transcription factor according to any one of claims 1 to 18, or first nucleic acid encoding said transcription factor; and
(ii) a second nucleic acid which comprises a target nucleotide sequence to be transcribed operatively linked to an HNF1-dependent promoter comprising an HNF1 binding site.

34. A composition according to claim 33 comprising said first nucleic acid.

35. A composition according to claim 34 wherein said first nucleic acid encodes a fusion protein comprising the transcriptional activator or repressor domain and the DNA-binding domain, wherein said fusion protein optionally comprises said regulatory domain.

36. A composition according to claim 34 wherein said first nucleic acid encodes a fusion protein comprising the DNA-binding domain and the regulatory domain, which fusion protein associates with a transcriptional activator or repressor domain to form the transcription factor.

37. A composition according to claim 36 wherein said first nucleic acid comprises separate sequences encoding (i) a fusion protein which comprises said DNA-binding domain and the regulatory domain and (ii) a polypeptide that associates with the fusion protein to provide the transcription factor.

38. A composition according to claim 37 wherein said separate sequences are within separate nucleic acid molecules.

39. A composition according to any one of claims 34 to 38 wherein said first and second nucleic acids are separate molecules.

40. A host cell comprising a composition according to any one of claims 33 to 39.
